(19) United States
(12) Patent Application Publication
(10) Pub. No.: US 2023/0233359 A1
(43) Pub. Date: Jul. 27, 2023

(54) TONGUE SUPPRESSION SYSTEM

(71) Applicant: TIMOTHY F. KELLEY, Newport Beach, CA (US)

(72) Inventor: TIMOTHY F. KELLEY, Newport Beach, CA (US)

(21) Appl. No.: 17/648,955

(22) Filed: Jan. 26, 2022

(51) Int. Cl. A61F 5/56

(52) U.S. Cl. CPC A61F 5/566

(57) ABSTRACT

A procedure for minimizing snoring and apnea in a human by properly positioning the tongue, the procedure being done using local anesthesia, and allowing for subsequent adjustment of tension on device both immediate and delayed.

### BACKGROUND OF THE INVENTION

### 1. Field of the Invention

A system for suspending the human tongue and tongue base in a way that allows for normal tongue function while preventing the tongue from becoming abnormally positioned in the airway during sleep, thus significantly reducing snoring and sleep apnea.

### 2. Description of the Prior Art

A number of techniques for reducing snoring and sleep apnea by correcting the abnormal tongue position in a human subject has been disclosed in the prior art. For example, U.S. Pat. No. 5,988,171 to Sohn et al discloses a technique for the treatment of human airway obstruction, sleep apnea and snoring by inserting a bone anchor into the mandible of the patient and fastening a suture to the bone anchor to suspend the tongue from the mandible. U.S. Pat. No. 8,973,582 to Rousseau discloses an implantable tissue suspension device and its method for use, the device including first and second filamentary elements bonded together only at a central connection region such that first and second leading and trailing legs of the respective first and second filamentary elements extend outwardly from the central connection region. The device is then implanted such that the central connection region extends laterally across a patient's tongue and the first and second leading and trailing legs of the first and second filamentary elements extend through the tongue such that ends thereof are positioned external to the genioglossus muscle. Currently, devices in existence are placed through incisions with a measure of soft tissue dissection that requires the patient to be under general anesthesia.

What is desired is to provide a procedure for significantly reduced human snoring and sleep apnea without the use of general anesthetic through a percutaneous approach and wherein the tongue suspension effect can be maintained over the usual time period.

### SUMMARY OF THE INVENTION

The present invention provides a method and apparatus for the treatment of snoring and sleep apnea by suspending the tongue and tongue base that allows for normal tongue function but prevents the tongue from becoming abnormally positioned in the patient's airway. This abnormal tongue positioning is a major contributing factor in snoring and sleep apnea.

The device of the present invention is composed of a multi prong, star shaped metal alloy (nitinal) soft tissue engagement piece that is attached to a thin cord of wire or suture that is placed percutaneously through an introducing trocar. This introducing trocar is a small-bore metal tube with a sharpened tip (needle) and is guided to the proper position in the tongue through an introduction site adjacent to the genial tubercle of the mandible. This guided introduction is controlled for accuracy by the premeasured length of the trocar and the skill and training of the operator. Once properly positioned, the device is deployed from the introducing trocar into position in the tongue by a deployment shaft that is slightly smaller than the bore of the introducing trocar. The introducing trocar is then removed, leaving the metal tissue engagement piece with attached suspension cord. Proper tension is then applied to the suspension cord to engage the multi prong nitinol tissue engagement piece. Once properly engaged, the deployment shaft is disconnected and removed leaving the engaged multi prong metal tissue engagement piece with attached cord in the tongue with the suspension cord exiting the introduction site. The suspension cord anchoring end is then securely attached to the mandible with a bore anchor device designed to allow for tightening of the suspension cord up to 6-12 months after initial placement. An alternative deployment technique is similar to the one just described but involves deployment of two metal engagement piece/suspension cord devices that are tied together around a mandibular bone bridge, thus avoiding placement of any mandible anchoring hardware. Another variation employs the use of “bathed suture” arranged in a way that allows the barbed suture to be pulled through the drilled bone canal such that the barbs spring open once the desired length and tension is reached thus “locking” the suture in place. An alternative technique involves introducing the device along the inferior border (lower border) of the mandible and not through a drilled channel. This would still allow for anchoring to the rigid periosteal covering of the mandible in this location.

It is believed that no device is currently available that suspends the tongue and tongue base through a percutaneous approach. It is also believed that no device is currently available that uses a flexible metal alloy soft tissue engagement device that directly connects to and “grabs” or firmly engages the soft tissue. Specifically, the devices in existence are placed through incisions with a measure of soft tissue dissection that requires the patient to be under general anesthesia. The device of the present invention improves on this in that it is placed through a local anesthesia approach, significantly reducing risk and minimizing soft tissue trauma. The vector of suspension currently used in existing devices is improved in that the vector of suspension for the device of the present invention is a direct, anterior-posterior vector with two points of fixation minimizing variability and direction of the suspension. The anterior anchor portion of the installed system can be accessed and tightened up to twelve months alter initial placement increasing the effective usefulness of the procedure and addressing the typical “suspension slackening” of prior suture suspension devices that rely entirely on simple suture soft tissue engagement. With simple suture soft tissue engagement, there is often a phenomenon of “cheese wiring” whereby the suture, under tension, slowly cuts through the soft tissue over time and with tissue movement, thereby slackening the tension on the soft tissue and reducing the suspension effect. This is avoided with the use of the more widely contacting and somewhat flexible metal alloy soft tissue engagement portion of the device.

### DESCRIPTION OF DRAWINGS

For a better understanding of the present invention as well as other objects and further features thereof, reference is made to the following description which is to be read in conjunction with the accompanying drawing wherein:

FIG. 1 illustrates the trocar, a luer lock fitting at the end of the trocar shaft, the deployment shaft exiting the luer lock end of the trocar, and the suture exiting the end of the deployment shaft;

FIG. 2 illustrates the deployment shaft exiting the front of the trocar with the metal tissue engagement piece at the leading edge of the deployment shaft;

FIG. 3 illustrates the deployment shaft with the metal tissue engagement piece at the leading edge and the suture passing through the shaft of the deployment shaft;

FIG. 4 illustrates the metal tissue engagement piece with attached suture;

FIG. 5 illustrates the initial part of the deployment process. Note the bone channel has already been drilled through the central portion of the mandible at the level of the genial tubercle. The Trocar is at the introduction site and the deployment shaft with attached metal tissue engagement piece and suture are contained within the trocar;

FIG. 6 illustrates the Trocar/deployment shaft/metal tissue engagement piece/suture complex passing through the drilled bone channel in the mandible and into the soft tissue of the tongue at the predetermined position so as not to pass completely through the tongue;

FIG. 7 illustrates the Trocar/deployment shaft/metal tissue engagement piece/suture at its predetermined position;

FIG. 8 illustrates the deployment shaft with attached metal tissue engagement piece now pushed through the shaft with the metal tissue engagement piece “sprung” open;

FIG. 9 illustrates the deployment shaft with attached metal engagement piece after the Trocar has been removed; and

FIG. 10 illustrates the metal tissue engagement piece after the deployment shaft has been detached. The metal tissue engagement piece with suture is now ready for final anchoring to the drilled bone channel to be then hence fully deployed.

### DESCRIPTION OF THE INVENTION

In accordance with the first step of the present invention, an incision is made in the chin at a location along the lower front edge at a position referred to as the submental crease. The soft tissue overlying the mandible in this area is then dissected off. A channel is then drilled through the mandible or alternatively the Trocar/deployment shaft/metal tissue engagement piece/suture is inserted along the lower edge of the mandible through rigid periosteum. The next series of steps are outlined and illustrated as per the figures above.

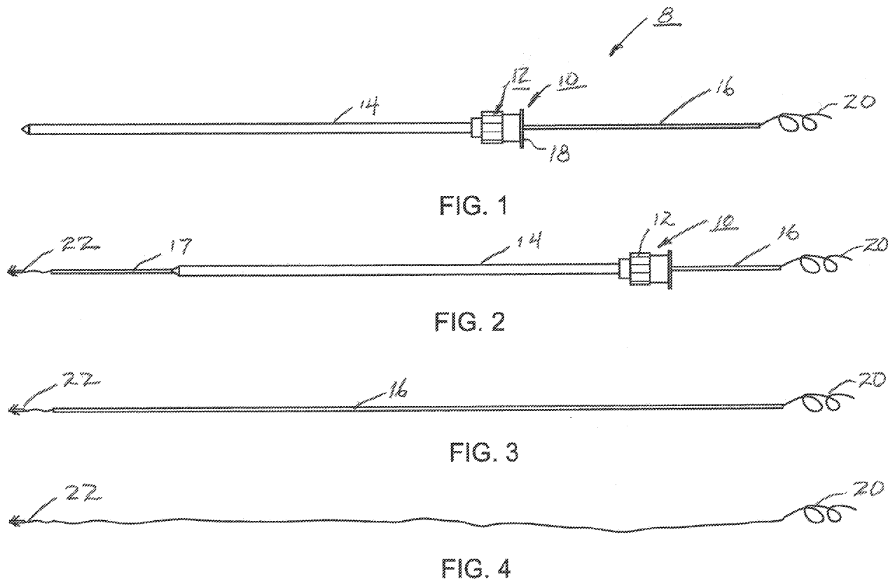

Referring to FIG. 1, the components which comprise the tongue suspension of the present invention is illustrated. In particular, system 8 compromises a trocar 10 having a lure locking fitting 12 at one and shaft 4 deployment shaft 16 exiting the lure lock and 18 of trocar 10 and suture 20 exiting the other end of tracer 10.

FIG. 2 illustrates a portion of deployment shaft 17 exiting the front of trocar 10 with a metal tissue engagement piece 22 positioned at the leading edge of shaft portion 17.

FIG. 3 illustrates deployment shaft 16 with metal engagement piece 22 at the leading edge of suture 20, suture 20 having passed through deployment shaft 16.

FIG. 4 illustrates metal engagement piece 22 attached to the suture end after deployment shaft 16 has been removed from the tongue suppression system 8.

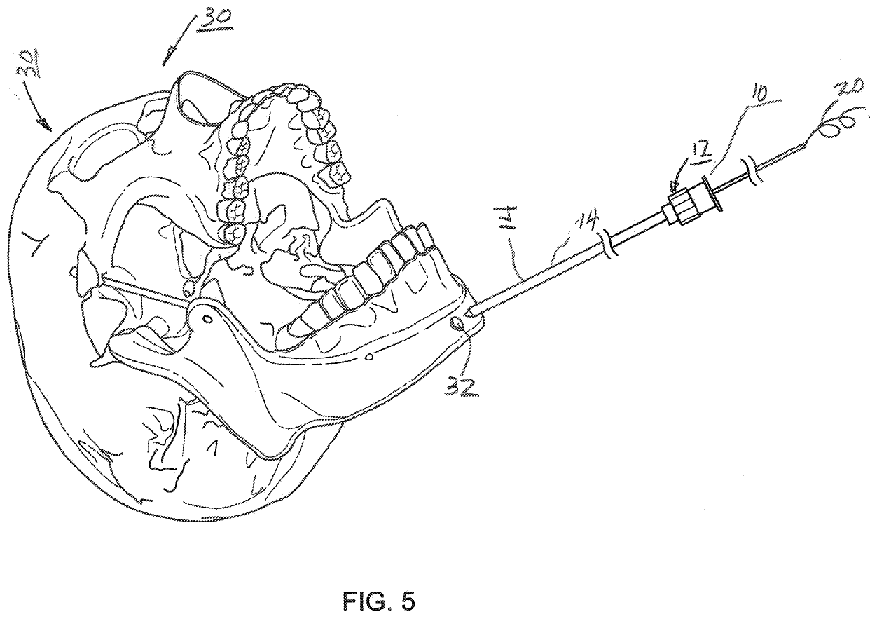

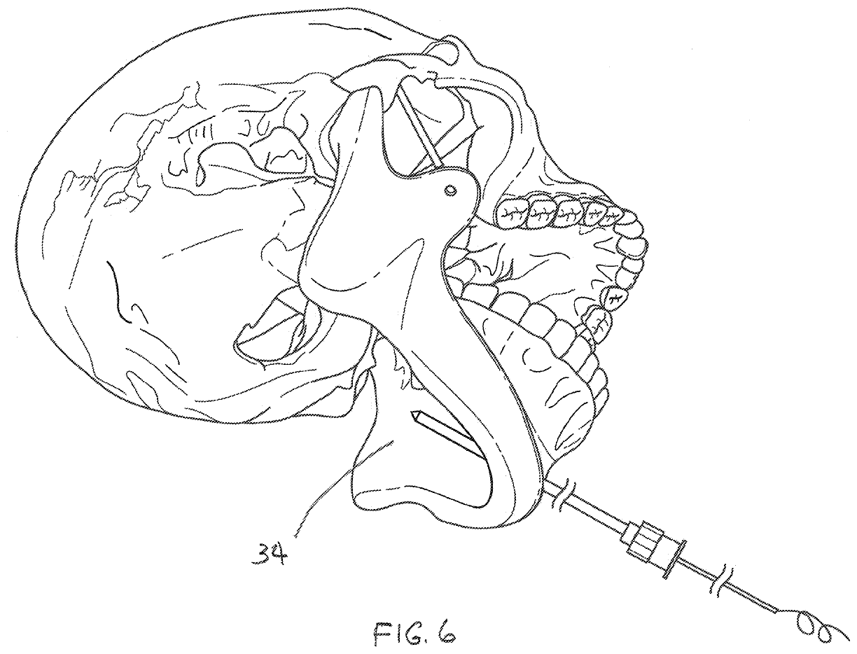

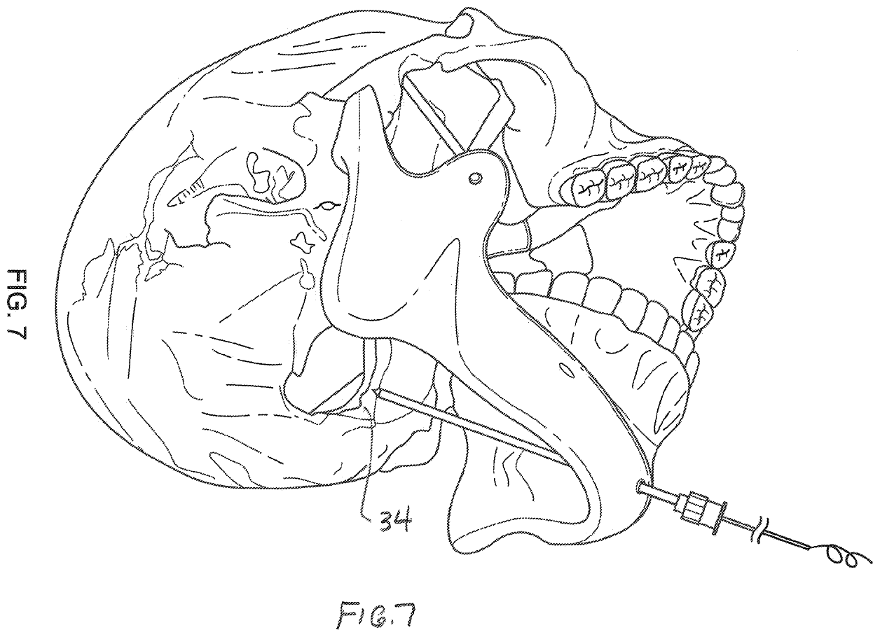

FIGS. 5-10 illustrate the steps used in the method of the present invention. In particular, FIG. 5 is an internal view of a human skull 30 and is the initial step in the suture deployment, process (a channel in the bone has already been drilled through the central portion of the mandible at the level of the genial tubercle, opening 32 being the introduction site). At this point, trocar 10 is the same structure as shown in FIG. 1.

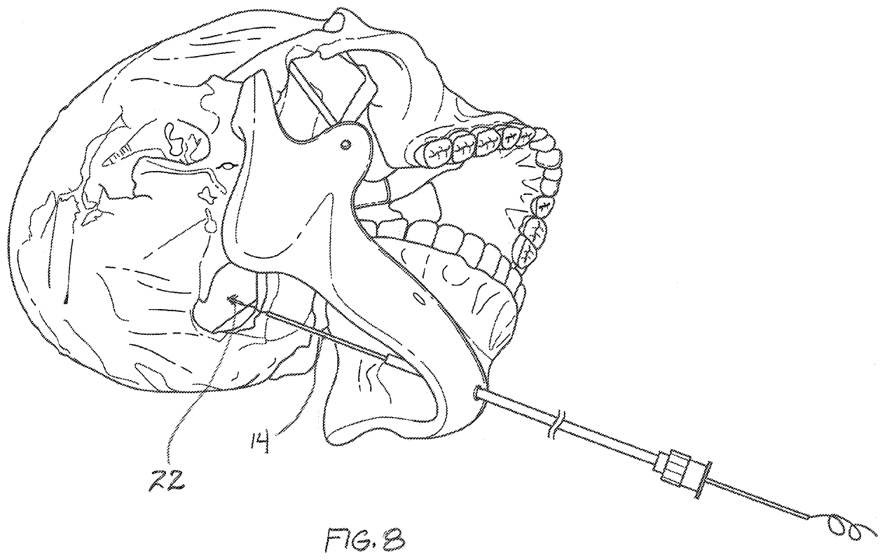

FIG. 6 illustrates the system comprising trocar 10, deployment shaft 14, metal tissue engagement piece 22 and suture 20 passing through the drilled bone channel formed in the mandible into the soft tissue of tongue 34 at a predetermined position such that the tongue 34 is not completely pierced.

FIG. 7 illustrates when the device has reached the desired position.

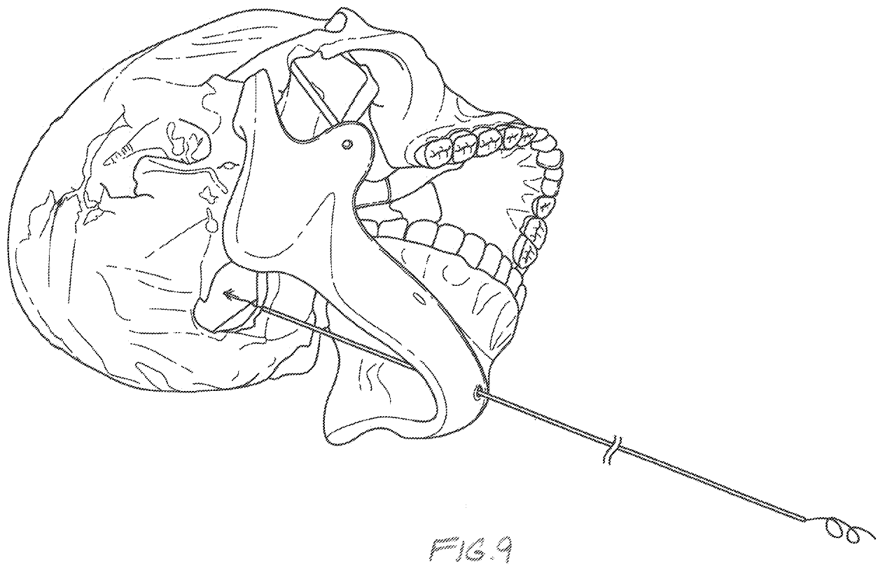

FIG. 8 illustrates deployment shaft 14 with attached metal tissue engagement piece 22 pushed through deployment shaft 14. At this position, the health provider causes metal tissue engagement piece 22 to open (originally in the closed, or compact position).

FIG. 9 illustrates deployment shaft 14 with the attached engagement piece after trocar 10 has been removed.

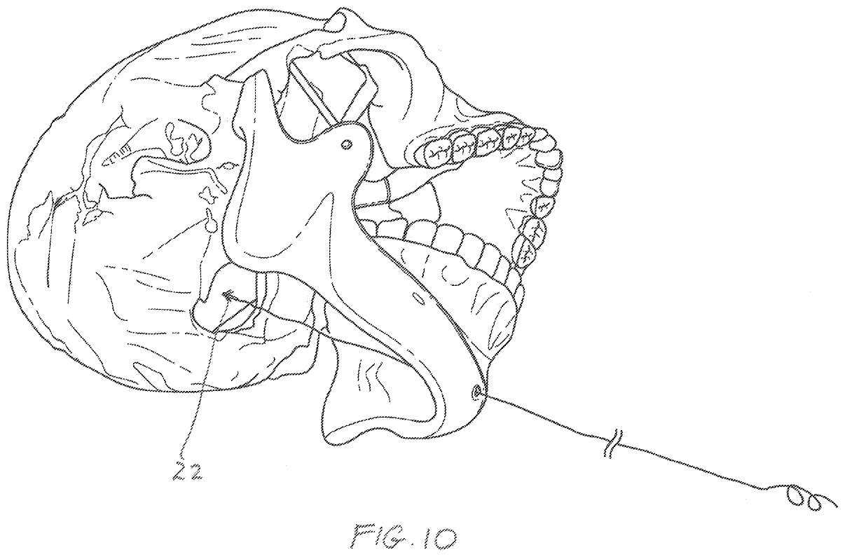

FIG. 10 illustrates metal tissue engagement piece 22 after deployment shaft 14 has been detached and removed leaving the metal tissue engagement piece 22 ready for final anchoring to the drilled bone channel fully deployed.

In summary, the device of the present invention is composed of a multi-prong, “star-shaped” metal alloy (nitinal) soft tissue engagement piece that is attached to a thin cord of wire or suture that is placed percutaneously through an introducing trochar. This introducing trochar is small bore metal tube with a sharpened tip, similar to a large needle. It is guided to the proper position in the tongue through an introduction site adjacent to the genial tubercle of the mandible. This guided introduction is controlled for accuracy by the introducing trochar's pre-measured length and the skill and training of the operator. Once properly positioned, the device is deployed from the introducing trochar into position in the tongue by a deployment shaft that is slightly smaller than the bore of the introducing trocar. The introducing trochar is then removed, leaving the metal engagement piece with attached suspension cord and the deployment shaft. Proper tension is then applied to the suspension cord and deployment shaft to engage the multi-prong nitinol tissue engagement piece. Once properly engaged, the deployment shaft is disconnected and removed. This leaves the engaged multi-prong metal piece with attached cord in the tongue with the suspension cord anchoring end exiting the introduction site. The suspension cord anchoring end is then securely attached to the mandible with a bone anchor device. This bone anchor device is designed to allow for tightening of the suspension cord 6-12 months after initial placement. Alternatively, a second device may be deployed just under the trajectory of the first device, and with a slightly different vector, then tied to the first device cord across a mandible bone bridge.

While the invention has been described with reference to its preferred embodiment, it will be understood by those skilled in the art that various changes may be made and equivalents may be substituted for elements thereof without departing from the true spirit and scope of the invention. In addition, many modifications may be made to adapt a particular situation or material to the teachings of the invention without departing from its essential teachings.

What is claimed is:

1. A method for treating snoring and sleep apnea caused by the position of the tongue of a human comprising the steps of:
providing a trocar device;
introducing an engagement piece that is attached to a thin cord into the trocar;
guiding said trocar to a predetermined position within said tongue,
deploying said engagement piece from said trochar,
removing said trochar from the introduction site, leaving in place said engagement piece with the attached suspension cord and deployment shaft;
applying tension to said suspension cord thus engaging said tissue engagement piece; and
removing said deployment shaft, said multi prong metal piece with attached cord remaining in the tongue, one end of the extension cord exiting the introduction site, the other end of the suspension cord being attached to the mandible.

2. The method of claim 1 wherein said treatment is performed under local anesthesia.

3. The method of claim 2 wherein said tension is adjustable such that the initial position of the tongue is substantially maintained over a predetermined time period.

4. A device for treating snoring and sleep apnea caused by the position of the tongue of a human comprising;
a trocar having a hollow center and a shaft extending from a first end of said trocar;
a deployment shaft movable within the hollow center of said trocar and having first and second ends;
a member attached to a first end of said deployment shaft adapted for engagement by a user; and
an anchor member attached to the second end of said deployment shaft.
